(19) United States
(12) Patent Application Publication
(10) Pub. No.: US 2026/0184686 A1
(43) Pub. Date: Jul. 2, 2026

(54) IMPROVING YIELD OF HYDROXYMETHYLFURFURAL THROUGH PURIFICATION OF SUGAR FEED

(71) Applicant: UOP LLC, Rosemont, IL (US)

(72) Inventors: Erin M. Broderick, Arlington Heights, IL (US); Gregory B. Kuzmanich, Arlington Heights, IL (US); Dean Emilio Rende, Arlington Heights, IL (US)

(21) Appl. No.: 19/418,307

(22) Filed: Dec. 12, 2025

Related U.S. Application Data
(60) Provisional application No. 63/740,641, filed on Dec. 31, 2024.

(51) Int. Cl. C07D 307/42

(52) U.S. Cl. CPC C07D 307/42

(57) ABSTRACT

Hydroxymethylfurfural (HMF) is derived from C6 sugars and is a versatile platform chemical with the potential to replace a range of conventional building blocks. It has been found that the remove of certain components such as oxygenated contaminants from C6 sugars can significantly improve the yields of HMF. The oxygenated contaminants may be removed by ion exchange resins, adsorbents, solvent extraction and pH adjustment via addition of base.

### FIELD

This disclosure relates to a process for increased yield of hydroxymethylfurfural from sugar feeds through the removal of oxygenates from sugar containing feeds.

### BACKGROUND OF THE INVENTION

With an increasing trend of sustainability across the globe, natural resources are being considered as feedstocks for fuels and petrochemicals. Sugars are a possible renewable resource with 177 MMT produced globally. Hydroxymethylfurfural (HMF) is derived from C6 sugars and is a versatile platform chemical with the potential to replace a range of conventional building blocks. HMF can be converted to paraxylene (PX), dimerized to produce linear olefins for linear alkyl benzenes (LAB), or dimerized to produce a feed for sustainable aviation fuel (SAF).

### SUMMARY OF THE INVENTION

A process of synthesizing hydroxymethylfurfural is provided, comprising removing, converting or neutralizing oxygen-containing contaminants from a feed comprising biomass-derived cellulose, or a sugar monomer or oligomer, or a mixture thereof to produce a purified feed and reacting the purified feed under reaction conditions with a catalyst to produce said hydroxymethylfurfural. The catalyst may be a heterogeneous catalyst such as a zeolite or a metal phosphate or a homogeneous catalyst. The feed may contain C5 and/or C6 sugars and organic acids, water, salt and an organic solvent. The contaminants that are removed include acids and salts. It has been found that the reaction to produce HMF is more selective when the acids, such as acetic acid, are first removed from the feed.

### BRIEF DESCRIPTION OF THE DRAWING

FIG. 1 shows the selectivity to hydroxymethylfurfural at different concentrations of acetic acid in the aqueous solution.

### DETAILED DESCRIPTION

A process of synthesizing hydroxymethylfurfural is provided that comprises removing, converting or neutralizing oxygen-containing contaminants from a feed comprising biomass-derived cellulose, or a sugar monomer or oligomer, or a mixture thereof to produce a purified feed and reacting the purified feed under reaction conditions with a catalyst to produce said hydroxymethylfurfural.

The removal of the contaminants is done at a temperature from about 25° C. to 100° C. The oxygen-containing contaminants include oxygenates with hydroxyl functional groups. One of the most frequently found contaminants is acetic acid. The contaminants are removed by an ion exchange resin, an adsorbent, or by solvent extraction. When the contaminants are acids, they may be neutralized to about a neutral pH by addition of a base. The ion exchange resin that is used is a basic anion exchange resin. The adsorbents are selected from activated carbon, zeolites and metal-organic frameworks. The solvent extraction is by contact of the contaminant with an organic solvent. The organic solvent may be selected from ethyl acetate, methyl tert-butyl ether, ionic liquids, diethyl ether, chloroform and dichloromethane. The base may be selected from bases having cations comprising sodium, potassium, lithium or cations and hydroxide or carbonate anions.

About 1-99 wt % and preferably about 75-99 wt % of the contaminant is removed from the feed. The catalyst may be a heterogeneous catalyst or a homogeneous catalyst. The heterogeneous catalyst may be a zeolite or a metal phosphate and the homogeneous catalyst may be sulfuric acid or hydrochloric acid. The sugar may be a C6 or a C5 sugar. These sugars may be derived from cellulose. The feed may further comprise water and optionally an organic solvent. The organic solvent may comprise a cyclic ether, an alcohol, a sulfoxide, a ketone, or combinations thereof. The feed may comprise 1 wt % to 50 wt % sugar. In addition, the feed may comprise from 1 wt % to 20 wt % of a salt comprising sodium chloride, lithium chloride, potassium chloride, cesium chloride, magnesium chloride, calcium chloride, or combinations thereof. The process may be a continuous process, a semi-continuous process or a batch process. The feed has a pH between about 4.5 and 7 after treatment. In addition, there is provided a process of synthesizing hydroxymethylfurfural comprising removing, converting or neutralizing oxygenates from a feed comprising biomass-derived cellulose, or a sugar monomer or oligomer, or a mixture thereof to produce a purified feed and reacting the purified feed under reaction conditions with a catalyst to produce said hydroxymethylfurfural. There is also provided a process of synthesizing hydroxymethylfurfural comprising removing, converting or neutralizing acids from a feed comprising biomass-derived cellulose, or a sugar monomer or oligomer, or a mixture thereof to produce a purified feed and reacting the purified feed under reaction conditions with a catalyst to produce said hydroxymethylfurfural.

### EXAMPLE

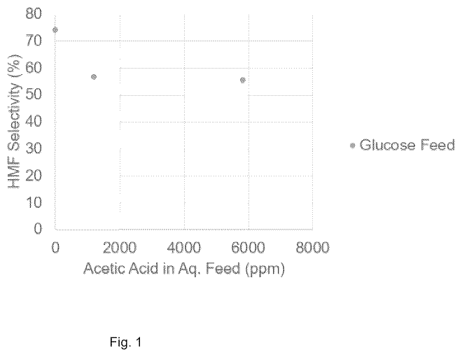

Batch autoclave experiments were performed in a 75 mL autoclave with a salt bath. In a typical experiment, the sugar (0.4 g of glucose), acetic acid, and sodium chloride (0.8 g) were dissolved in water (4 g). The water solution was loaded into the autoclave followed by the solvent tetrahydrofuran (THF, 14 g). The catalyst (40-60 mesh of ZrO(PO₄)₂, 0.094 g) was then added to the autoclave, which was sealed and placed into the salt bath. After stirring for a set amount of time, the autoclave was removed from the salt bath and placed into an ice bath for 30 min. The solution was centrifuged to separate the liquid from the solid phases. The organic and aqueous phases were also decanted via a pipette. Samples were submitted for both phases for analysis. A significantly higher yield of HMF was found in samples which were treated to remove acetic acid. The effect of the amount of acetic acid in the feed is shown in FIG. 1.

### SPECIFIC EMBODIMENTS

While the following is described in conjunction with specific embodiments, it will be understood that this description is intended to illustrate and not limit the scope of the preceding description and the appended claims.

A first embodiment of the invention is a process of synthesizing hydroxymethylfurfural comprising removing, converting or neutralizing oxygen containing contaminants from a feed comprising biomass-derived cellulose, or a sugar monomer or oligomer, or a mixture thereof to produce a purified feed and reacting the purified feed under reaction conditions with a catalyst to produce the hydroxymethylfurfural. An embodiment of the invention is one, any or all of prior embodiments in this paragraph up through the first embodiment in this paragraph wherein the removal of the contaminants is done at a temperature from about 25° C. to 100° C. An embodiment of the invention is one, any or all of prior embodiments in this paragraph up through the first embodiment in this paragraph wherein the oxygen containing contaminants have hydroxyl functional groups. An embodiment of the invention is one, any or all of prior embodiments in this paragraph up through the first embodiment in this paragraph wherein the contaminant is acetic acid. An embodiment of the invention is one, any or all of prior embodiments in this paragraph up through the first embodiment in this paragraph wherein the contaminants are removed by an ion exchange resin, an adsorbent, or by solvent extraction. An embodiment of the invention is one, any or all of prior embodiments in this paragraph up through the first embodiment in this paragraph wherein the contaminants are acids that are neutralized to about a neutral pH by addition of a base. An embodiment of the invention is one, any or all of prior embodiments in this paragraph up through the first embodiment in this paragraph wherein the ion exchange resin comprises a strong base ionic exchanger. An embodiment of the invention is one, any or all of prior embodiments in this paragraph up through the first embodiment in this paragraph wherein the adsorbent is selected from activated carbon, zeolites and metal organic frameworks. An embodiment of the invention is one, any or all of prior embodiments in this paragraph up through the first embodiment in this paragraph wherein the solvent extraction is by contact of the contaminant with a solvent selected from ethyl acetate, methyl tert-butyl ether, ionic liquids, diethyl ether chloroform and dichloromethane. An embodiment of the invention is one, any or all of prior embodiments in this paragraph up through the first embodiment in this paragraph wherein the base is selected from bases having cations comprising sodium, potassium, lithium or cations and hydroxide or carbonate anions. An embodiment of the invention is one, any or all of prior embodiments in this paragraph up through the first embodiment in this paragraph wherein about 1-99 wt % of the contaminant is removed from the feed. An embodiment of the invention is one, any or all of prior embodiments in this paragraph up through the first embodiment in this paragraph wherein about 75-99 wt % of the contaminant is removed from the feed. An embodiment of the invention is one, any or all of prior embodiments in this paragraph up through the first embodiment in this paragraph wherein the catalyst is selected from a heterogeneous catalyst or a homogeneous catalyst. An embodiment of the invention is one, any or all of prior embodiments in this paragraph up through the first embodiment in this paragraph wherein the heterogeneous catalyst is a zeolite or a metal phosphate. An embodiment of the invention is one, any or all of prior embodiments in this paragraph up through the first embodiment in this paragraph wherein the homogeneous catalyst is sulfuric acid or hydrochloric acid. An embodiment of the invention is one, any or all of prior embodiments in this paragraph up through the first embodiment in this paragraph wherein the sugar monomer is a C6 or a C5 sugar. An embodiment of the invention is one, any or all of prior embodiments in this paragraph up through the first embodiment in this paragraph wherein the sugars are derived from cellulose. An embodiment of the invention is one, any or all of prior embodiments in this paragraph up through the first embodiment in this paragraph wherein the feed further comprises water and optionally an organic solvent. An embodiment of the invention is one, any or all of prior embodiments in this paragraph up through the first embodiment in this paragraph wherein the organic solvent comprises a cyclic ether, an alcohol, a sulfoxide, a ketone, or combinations thereof. An embodiment of the invention is one, any or all of prior embodiments in this paragraph up through the first embodiment in this paragraph wherein the feed comprises 1 wt % to 50 wt % sugar. An embodiment of the invention is one, any or all of prior embodiments in this paragraph up through the first embodiment in this paragraph wherein the feed comprises from 1 wt % to 20 wt % of a salt comprising sodium chloride, lithium chloride, potassium chloride, cesium chloride, magnesium chloride, calcium chloride, or combinations thereof. An embodiment of the invention is one, any or all of prior embodiments in this paragraph up through the first embodiment in this paragraph wherein the process is a continuous process, a semi-continuous process or a batch process. An embodiment of the invention is one, any or all of prior embodiments in this paragraph up through the first embodiment in this paragraph wherein the feed has a pH between about 4.5 and 7.

A second embodiment of the invention is a process of synthesizing hydroxymethylfurfural comprising removing, converting or neutralizing oxygenates from a feed comprising biomass-derived cellulose, or a sugar monomer or oligomer, or a mixture thereof to produce a purified feed and reacting the purified feed under reaction conditions with a catalyst to produce the

### Hydroxymethylfurfural.

A third embodiment of the invention is a process of synthesizing hydroxymethylfurfural comprising removing, converting or neutralizing acids from a feed comprising biomass-derived cellulose, or a sugar monomer or oligomer, or a mixture thereof to produce a purified feed and reacting the purified feed under reaction conditions with a catalyst to produce the hydroxymethylfurfural.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of synthesizing hydroxymethylfurfural comprising removing, converting or neutralizing oxygen containing contaminants from a feed comprising biomass-derived cellulose, or a sugar monomer or oligomer, or a mixture thereof to produce a purified feed and reacting said purified feed under reaction conditions with a catalyst to produce said hydroxymethylfurfural.

2. The process of claim 1 wherein said removal of said contaminants is done at a temperature from about 25° C. to 100° C.

3. The process of claim 1 wherein said oxygen containing contaminants have hydroxyl functional groups.

4. The process of claim 1 wherein said contaminant is acetic acid.

5. The process of claim 1 wherein said contaminants are removed by an ion exchange resin, an adsorbent, or by solvent extraction.

6. The process of claim 1 wherein said contaminants are acids that are neutralized to about a neutral pH by addition of a base.

7. The process of claim 5 wherein said adsorbent is selected from activated carbon, zeolites and metal organic frameworks.

8. The process of claim 5 wherein said solvent extraction is by contact of said contaminant with a solvent selected from ethyl acetate, methyl tert-butyl ether, ionic liquids, diethyl ether chloroform and dichloromethane.

9. The process of claim 1 wherein about 1-99 wt % of said contaminant is removed from said feed.

10. The process of claim 1 wherein said catalyst is selected from a heterogeneous catalyst or a homogeneous catalyst.

11. The process of claim 10 wherein said heterogeneous catalyst is a zeolite or a metal phosphate.

12. The process of claim 10 wherein said homogeneous catalyst is sulfuric acid or hydrochloric acid.

13. The process of claim 1 wherein said sugar monomer is a C6 or a C5 sugar.

14. The process of claim 1 wherein said sugars are derived from cellulose.

15. The process of claim 1 wherein said feed further comprises water and optionally an organic solvent.

16. The process of claim 1 wherein said organic solvent comprises a cyclic ether, an alcohol, a sulfoxide, a ketone, or combinations thereof.

17. The process of claim 1 wherein said feed comprises from 1 wt % to 20 wt % of a salt comprising sodium chloride, lithium chloride, potassium chloride, cesium chloride, magnesium chloride, calcium chloride, or combinations thereof.

18. The process of claim 1 wherein said feed has a pH between about 4.5 and 7.

19. A process of synthesizing hydroxymethylfurfural comprising removing, converting or neutralizing oxygenates from a feed comprising biomass-derived cellulose, or a sugar monomer or oligomer, or a mixture thereof to produce a purified feed and reacting said purified feed under reaction conditions with a catalyst to produce said hydroxymethylfurfural.

20. A process of synthesizing hydroxymethylfurfural comprising removing, converting or neutralizing acids from a feed comprising biomass-derived cellulose, or a sugar monomer or oligomer, or a mixture thereof to produce a purified feed and reacting said purified feed under reaction conditions with a catalyst to produce said hydroxymethylfurfural.
